(19) United States
(12) Patent Application Publication
(10) Pub. No.: US 2022/0011219 A1
(43) Pub. Date: Jan. 13, 2022

(54) DISEASE DIAGNOSIS USING SPECTROSCOPY AND MACHINE LEARNING

(71) Applicants: Massachusetts Institute of Technology, Cambridge, MA (US); Mohammed VI Polytechnic University, Ben Guerir (MA); Laboratoire Anoual, Casablanca (MA)

(72) Inventors: Dimitris Bertsimas, Belmont, MA (US); Driss Lahlou Kitane, Somerville, MA (US); Nawfel Azami, Rabat (MA); Jamal Fekkak, Casablanca (MA); Rachid Benhida, Nice (FR); Salma Loukman, Rabat (MA); Nabila Marchoudi, Casablanca (MA)

(73) Assignees: Massachusetts Institute of Technology, Cambridge, MA (US); Mohammed VI Polytechnic University, Ben Guerir (MA); Laboratoire Anoual, Casablanca (MA)

(21) Appl. No.: 17/368,803

(22) Filed: Jul. 6, 2021

Related U.S. Application Data
(60) Provisional application No. 63/048,869, filed on Jul. 7, 2020.

(51) Int. Cl. G01N 21/35; G06N 20/00; G06F 17/18; G16H 50/20; G16H 70/60; G16H 50/70; G16H 10/60; G16H 10/40; G06K 9/62; G01N 33/483

(52) U.S. Cl. CPC G01N 21/35; G06N 20/00; G06F 17/18; G16H 50/20; G16H 70/60; G16H 50/70; G01N 2201/06113; G16H 10/40; G06K 9/6257; G06K 9/6247; G06K 9/6265; G01N 33/483; G16H 10/60

(57) ABSTRACT

Aspects of the present application relate to techniques of diagnosing whether a pathogen (e.g., SARS-CoV-2) is present in a subject using infrared (IR) spectroscopy and machine learning techniques. The techniques use spectral data obtained from performing IR spectroscopy on a biological sample (e.g., saliva or nasal sample, or genetic material extracted therefrom) to generate a set of feature values. The feature values are provided as input to a machine learning model to obtain output indicating whether the pathogen is present in the biological sample. The output of the machine learning model may be used to determine a diagnosis result for a subject.

### RELATED APPLICATIONS

This application claims priority under 35 U.S.C. § 119(e) to U.S. Provisional Application 63/048,869 entitled, “METHOD FOR DETECTING A PATHOGEN IN A HUMAN SAMPLE USING INFRARED SPECTROSCOPY,” filed Jul. 7, 2020, the entire contents of which is incorporated by reference herein.

### FIELD

This application relates generally to techniques of diagnosing a disease (e.g., COVID-19) using spectroscopy and machine learning. Techniques described herein generate a set of features using spectral data obtained from performing spectroscopy (e.g., infrared (IR) spectroscopy) on a biological sample from a subject, and provide the set of features as input to a machine learning model to obtain output indicating whether the subject has a disease.

### BACKGROUND

According to the World Health Organization (WHO), a pandemic is the worldwide spread of a new disease, characterized by a rapid propagation and high mortality rate. Transmitted by viruses, bacteria, and other pathogens, it kills millions of people. Several pandemics are well-known in human history, from various plagues in the Middle Ages to the Spanish influenza pandemic in the last century, and the more recent H1N1 type virus.

Presently, the world is experiencing an unprecedented health crises with the spread of SARS-CoV-2 virus (also referred to as “COVID-19”) around the world. The virus, which is believed to originally have appeared in Wuhan China in December 2019, rapidly spread all over the world in only a few weeks. The fast spread of COVID-19 is mainly attributed to the mode of transmission of the virus and high volume of international travel. Moreover, emerging mutations of the COVID-19 virus (also referred to as “COVID-19 variants”) have increased transmissibility and increased ability to escape the human immune system. The number of infected people is still increasing, with more than 140 million confirmed cases and more than 3 million confirmed deaths worldwide, after only one year.

Even with significant medical resources in the developed world, most sophisticated healthcare systems are being overwhelmed by the magnitude of the pandemic. Unfortunately, without available treatment, slowing the spread of the virus consists only in adopting social rules such as confinement, social distancing, limiting travel, cancelling large gatherings, etc. From limited healthcare workers to the lack of medical capacity, many countries are facing unprecedented health challenges in managing COVID-19.

### SUMMARY

Aspects of the present application relate to techniques of diagnosing whether a pathogen (e.g., SARS-CoV-2) is present in a subject using infrared (IR) spectroscopy and machine learning techniques. The techniques use spectral data obtained from performing IR spectroscopy on a biological sample (e.g., a saliva, nasal, skin, blood, urine, or fecal sample, or a genetic material extraction thereof) to generate a set of feature values. The feature values are provided as input to a machine learning model to obtain output indicating whether the pathogen is present in the biological sample. The output of the machine learning model may be used to determine a diagnosis result for a subject.

According to some embodiments, a disease diagnosis system is provided. The disease diagnosis system comprises: a spectrometer configured to perform infrared (IR) spectroscopy on a first biological sample from a subject to obtain spectral data comprising light intensity measurements for a plurality of wavelengths of light; a processor; and a non-transitory computer-readable storage medium storing instructions that, when executed by the processor, cause the processor to perform: generating, using the spectral data, a set of feature values for a subset of wavelengths of the plurality of wavelengths of light, wherein the subset of wavelengths indicate a spectral signature of a pathogen; and providing the set of feature values as input to a machine learning model to obtain output indicating whether the pathogen is present in the first biological sample from the subject. According to some embodiments, the pathogen is SARS-CoV-2.

According to some embodiments, the first biological sample comprises genetic material extracted from a second biological sample from the subject. According to some embodiments, the genetic material extracted from the second biological sample from the subject comprises an RNA extraction from the second biological sample. According to some embodiments, the first biological sample from the subject comprises a nasopharyngeal swab sample, a saliva sample, and/or a nasal sample.

According to some embodiments, the subset of wavelengths consists of less than 100 wavelengths. According to some embodiments, the subset of wavelengths is a set of wavelengths identified using mixed integer optimization. According to some embodiments, the machine learning model comprises a logistic regression model.

According to some embodiments, generating the set of feature values for the subset of wavelengths comprises: determining a second derivative of the spectral data; and determining the set of feature values for the subset of wavelengths to be values of the second derivative for the subset of the plurality of wavelengths. According to some embodiments, generating the set of feature values for the subset of wavelengths comprises: applying Savitzky-Golay filtering to obtained filtered spectral data; and determining the set of feature values for the subset of wavelengths using the filtered spectral data.

According to some embodiments, the spectrometer comprises an infrared (IR) Fourier transform (FT) spectrometer. According to some embodiments, the spectrometer is configured to perform spectroscopy on the biological sample to obtain measurements for wavelengths between approximately 600 cm−1 to 4500 cm−1. According to some embodiments, the spectrometer is configured to perform absorption, reflection, and/or transmission IR spectroscopy.

According to some embodiments, a method of determining whether a pathogen is present in a subject is provided. The method comprises: using a processor to perform: obtaining spectral data generated from performance of IR spectroscopy on a first biological sample from the subject, wherein the spectral data comprises light intensity measurements for a plurality of wavelengths of light; generating, using the spectral data, a set of feature values for a subset of wavelengths of the plurality of wavelengths of light, wherein the subset of wavelengths indicate a spectral signature of the pathogen; providing the set of feature values as input to a machine learning model to obtain output indicating whether the pathogen is present in the first biological sample from the subject. According to some embodiments, the pathogen is SARS-CoV-2.

According to some embodiments, the first biological sample comprises genetic material extracted from a second biological sample from the subject. According to some embodiments, the first biological sample from the subject is at least one of a group consisting of a nasopharyngeal swab sample, a saliva sample, and a nasal sample.

According to some embodiments, the subset of wavelengths consists of less than 100 wavelengths. According to some embodiments, the machine learning model comprises a logistic regression model. According to some embodiments, the plurality of wavelengths range from approximately 600 cm⁻¹ to 4500 cm⁻¹.

According to some embodiments, a non-transitory computer-readable storage medium storing instructions is provided. The instructions, when executed by a processor, causes the processor to perform: obtaining spectral data generated from performing IR spectroscopy on a first biological sample from the subject, wherein the spectral data comprises light intensity measurements for a plurality of wavelengths of light; generating, using the spectral data, a set of feature values for a subset of wavelengths of the plurality of wavelengths of light, wherein the subset of wavelengths indicate a spectral signature of a pathogen when a pathogen is present in a biological sample; and providing the set of feature values as input to a machine learning model to obtain output indicating whether the pathogen is present in the first biological sample from the subject. According to some embodiments, the pathogen may be SARS-CoV-2.

According to some embodiments, a system for diagnosing whether SARS-CoV-2 is present in a subject is provided. The system comprises: a spectrometer configured to perform IR spectroscopy on a first biological sample from the subject to obtain spectral data comprising light intensity measurements for a plurality of wavelengths of light; a processor; and a non-transitory computer-readable storage medium storing instructions that, when executed by the processor, cause the processor to perform: generating a set of feature values using the spectral data; and providing the set of feature values as input to a machine learning model to obtain output indicating whether SARS-CoV-2 is present in the first biological sample from the subject.

According to some embodiments, the first biological sample comprises genetic material extracted from a second biological sample from the subject. According to some embodiments, the first biological sample from the subject comprises a nasopharyngeal swab sample, a nasal sample, or a saliva sample.

According to some embodiments, the machine learning model comprises a logistic regression model. According to some embodiments, the spectrometer comprises an infrared (IR) Fourier transform (FT) spectrometer.

According to some embodiments, generating the set of feature values using the spectral data comprises generating a set of feature values with a number of dimensions less than a number of the plurality of wavelengths. According to some embodiments, generating the set of feature values comprises generating the set of feature values using one or more principal components identified from performing principal component analysis (PCA) or partial least squares regression (PLS).

According to some embodiments, a method for diagnosing whether SARS-CoV-2 is present in a subject is provided. The method comprises: using a processor to perform: obtaining spectral data generated from performance of IR spectroscopy on a first biological sample from the subject, wherein the spectral data comprises light intensity measurements for a plurality of wavelengths of light; generating a set of feature values using the spectral data; and providing the set of feature values as input to a machine learning model to obtain output indicating whether SARS-CoV-2 is present in the first biological sample from the subject.

According to some embodiments, a non-transitory computer-readable storage medium storing instructions is provided. The instructions, when executed by a processor, cause the processor to perform: obtaining spectral data generated from performance of IR spectroscopy on a first biological sample from the subject, wherein the spectral data comprises light intensity measurements for a plurality of wavelengths light; generating a set of feature values using the spectral data; and providing the set of feature values as input to a machine learning model to obtain output indicating whether SARS-CoV-2 is present in the first biological sample from the subject.

According to some embodiments, a method of training a machine learning model for diagnosing whether a pathogen is present in a subject is provided. The method comprises: using a processor to perform: obtaining spectral data obtained from performing IR spectroscopy on biological samples obtained from a plurality of subjects, wherein the spectral data comprises, for each of the plurality of subjects, light intensity measurements for a plurality of wavelengths of light; generating a set of training data using the spectral data; and training the machine learning model using the training data, the training comprising determining a set of features for the machine learning model, wherein the set of features has a number of dimensions that is less than a number of the plurality wavelengths.

According to some embodiments, determining the set of features comprises determining a subset of wavelengths of the plurality of wavelengths that indicate a spectral signature of the pathogen. According to some embodiments, determining the subset of the plurality of wavelengths to be the set of features comprises determining less than 100 of the plurality of wavelengths to be the set of features. According to some embodiments, the method further comprises determining the subset of wavelengths at least in part by performing mixed integer optimization to identify the subset of wavelengths.

According to some embodiments, determining the set of features comprises performing principal component analysis (PCA) to identify the set of features. According to some embodiments, determining the set of features comprises performing partial least square (PLS) regression to identify the set the features.

According to some embodiments, the method further comprises: obtaining diagnosis data comprising, for each of the plurality of subjects, an indication of whether the pathogen is determined to be present in the subject based on a different diagnosis technique; and generating the set of training data by using the diagnosis data to label sets of feature values for the at least some subjects.

According to some embodiments, the pathogen is SARS-CoV-2. According to some embodiments, the machine learning model comprises a logistic regression model. According to some embodiments, the plurality of wavelengths of light range from approximately 600 cm⁻¹ to 4500 cm⁻¹. According to some embodiments, the biological samples comprise extractions of genetic material.

According to some embodiments, determining the set of features for the machine learning model comprises: determining a second derivative of the spectral data; and determining the set of features using the second derivative values. According to some embodiments, processing the spectral data comprises applying Savitzky-Golay filtering to the spectral data.

According to some embodiments, a system of training a machine learning model for diagnosing whether a pathogen is present in a subject is provided. The system comprises: a processor; and a non-transitory computer-readable storage medium storing instructions, that when executed by the processor, causes the processor to perform: obtaining spectral data obtained from performing IR spectroscopy on biological samples obtained from a plurality of subjects, wherein the spectral data comprises, for each of the plurality of subjects, light intensity measurements for a plurality of wavelengths of light; and training the machine learning model using the spectral data, the training comprising determining a set of features for the machine learning model, wherein the set of features has a number of dimensions that is less than a number of the plurality wavelengths.

According to some embodiments, determining the set of features comprises determining a subset of wavelengths of the plurality of wavelengths that indicate a spectral signature of the pathogen. According to some embodiments, the instructions further cause the processor to perform identifying the subset of wavelengths at least in part by performing mixed integer optimization to identify the subset of wavelengths. According to some embodiments, the pathogen is SARS-CoV-2. According to some embodiments, the plurality of wavelengths range from approximately 600 cm⁻¹ to 4500 cm⁻¹. According to some embodiments, the biological samples comprise extractions of genetic material.

According to some embodiments, a non-transitory computer-readable storage medium storing instructions is provided. The instructions, when executed by a processor, cause the processor to perform a method to train a machine learning model for diagnosing whether a pathogen is present in a subject, the method comprising: obtaining spectral data obtained from performing IR spectroscopy on biological samples obtained from a plurality of subjects, wherein the spectral data comprises, for each of the plurality of subjects, light intensity measurements for a plurality of wavelengths of light; and training the machine learning model using the spectral data, the training comprising determining a set of features for the machine learning model, wherein the set of features has a number of dimensions that is less than a number of the plurality wavelengths.

The foregoing summary is provided by way of illustration and is not intended to be limiting. It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

FIG. 1A illustrates an example disease diagnosis system 100, according to some embodiments of the technology described herein.

FIG. 1B illustrates a data flow diagram in the inference system 106 of FIG. 1A, according to some embodiments of the technology described herein.

FIG. 1C illustrates an example of a training system 130 for training a machine learning model to obtain a trained machine learning model 106C used by the disease diagnosis system 100 of FIG. 1A, according to some embodiments of the technology described herein.

FIG. 2 is a diagram of an example process 200 for diagnosing COVID-19 in a subject, according to some embodiments of the technology described herein.

FIG. 3 is a flowchart of an example process 300 for diagnosing whether a pathogen is present in a subject, according to some embodiments of the technology described herein.

FIG. 4 is a flowchart of an example process 400 for diagnosing whether a pathogen is present in a subject, according to some embodiments of the technology described herein.

FIG. 5 is a flowchart of an example process 500 for training a machine learning model for diagnosing whether a pathogen is present in subject, according to some embodiments of the technology described herein.

FIG. 6A is a graph 600 plotting spectral data obtained from performing spectroscopy on a biological sample, according to some embodiments of the technology described herein.

FIG. 6B is a graph 602 of the data of graph 600 after undergoing pre-processing, according to some embodiments of the technology described herein.

FIG. 7A is a graph 700 of a subset of light wavenumbers of spectral data used to generate a set of feature values for input to a machine learning model, according to some embodiments of the technology described herein.

FIG. 7B is a table 710 listing chemical structures and/or processes associated with the light wavenumbers of FIG. 7A, according to some embodiments of the technology described herein.

FIG. 8A is a set of graphs of latent variables to use as feature values input to a machine learning model, according to some embodiments of the technology described herein.

FIG. 8B is a set of graphs of projections of the latent variables of FIG. 8A, according to some embodiments of the technology described herein.

FIG. 9 is an illustrative implementation of a computer system that may be used in connection with some embodiments of the technology described herein.

### DETAILED DESCRIPTION

The world is presently experiencing an unprecedented health crisis due to the appearance of the SARS-CoV-2 pathogen (also referred to as “COVID-19”). The pandemic has affected health, economies, and social life on a global scale. One of the main tools for controlling the spread of such a pandemic is having an efficient and reliable technique for diagnosing SARS-CoV-2 in subjects. Many areas of the world are unable to carry out the necessary level of testing to control the spread of the pathogen due to limitations in existing diagnostic techniques.

Conventional techniques of diagnosing the SARS-CoV-2 pathogen in a subject use a reverse transcription quantitative polymerase chain reaction (RT-PCR) to detect viral nucleic acids. The inventors have recognized conventional techniques require specialized handling of biological samples extracted from patients, require biological samples to be in an acute phase for reliable detection, and require a testing time that ranges from two to four hours. Moreover, conventional techniques require the use of expensive kits that are largely sourced from suppliers that may not be accessible to many countries during lockdown periods. As a result of these limitations, conventional techniques may take multiple days (e.g., 2, 3, 4, or 5 days) to return diagnosis results to a subject in some countries.

To address the limitations with conventional techniques of diagnosing the COVID-19 virus, the inventors have developed a more efficient and accessible diagnostic technique. The techniques described herein employ infrared (IR) spectroscopy (e.g., Fourier transform (FT) IR spectroscopy) and machine learning techniques to determine whether SARS-CoV-2 is present in a subject more efficiently than do conventional techniques. For example, the techniques described herein may be performed in a median time of approximately 1.5 minutes after extraction of RNA from a biological sample, whereas conventional RT-PCR based diagnosis techniques may take 2 to 4 hours after extraction of RNA. Moreover, techniques described herein do not require any reagents, and produce less biohazard waste than generated by conventional techniques.

Techniques described herein use spectral data obtained from performing IR spectroscopy on a biological sample (e.g., a saliva, nasal, skin, blood, urine, or fecal sample, or a genetic material extraction thereof) from a subject. For example, an IR spectrometer may be used to perform IR spectroscopy on the biological sample to measure the biological sample's reflectance, absorbance, or transmission of light applied to the biological sample. The techniques use the spectral data to generate a set of feature values that are provided as input to a machine learning model (e.g., logistic regression model, a support vector machine (SVM), neural network, etc.) trained to output an indication of whether a pathogen is present in the biological sample. For example, the machine learning model may be trained to output a classification of whether SARS-CoV-2 is present in the biological sample. The output of the machine learning model may be used to determine a diagnosis for a subject (e.g., to determine whether the subject is determined to be COVID-19 positive or negative).

Spectral data obtained from performing IR spectroscopy may have very high dimensionality because the spectral data includes light intensity values for thousands of wavelengths of light (e.g., wavenumbers). The inventors have recognized that the high dimensionality of the data may negatively impact performance (e.g., accuracy) of a machine learning model that uses the spectral data (e.g., as input features). Accordingly, the inventors have developed a machine learning model that takes as input a set of features with reduced dimensionality from that of the spectral data. For example, techniques described herein may reduce the thousands of light intensity measurements in a spectral data sample into a set of less than 100 values.

The inventors have further recognized that conventional techniques of dimension reduction provide a set of latent variables that may not provide a human interpretable indication of characteristics of a biological sample. For example, the latent variables obtained from performing principal component analysis (PCA) may not indicate physical phenomenon of a biological sample. Accordingly, the inventors have developed a machine learning model that uses a set of feature values (e.g., as input) that comprise of values determined for a subset of the wavelengths (e.g., wavenumbers) in the spectral data. For example, the set of feature values may be determined for less than 100 wavelengths of the spectral data (which may include measurements for thousands of wavelengths). Techniques described herein identify a subset of wavelengths that indicate a spectral signature of a pathogen (e.g., SARS-CoV-2). A machine learning model may be trained to determine whether the spectral signature is present based on the set of feature values for the subset of wavelengths. The subset of wavelengths may indicate characteristics of a biological sample which may, for example, allow a clinician to interpret a diagnosis result (e.g., by informing the clinician of chemical processes within the biological sample).

A spectrometer may also be referred to as a “spectrophotometer”, “spectrograph”, or “spectral analyzer”. In some embodiments, the spectrometer may be configured to perform absorbance spectroscopy, transmission spectroscopy, reflectance spectroscopy, diffusion spectroscopy, or other suitable type of spectroscopy. In some embodiments, the spectrometer may be configured to perform infrared (IR) spectroscopy. For example, the spectrometer may be configured to perform Fourier transform (FT) IR spectroscopy.

Spectral data obtained from performing spectroscopy on a biological sample may include light intensity measurements for multiple wavelengths of light applied during spectroscopy. A wavelength of light may be represented by a wavenumber (also referred to herein as “spatial frequency”) and/or a frequency. For example, spectral data obtained from performing absorbance spectroscopy may include intensity measurements of light absorbance for various light wavenumbers. In another example, spectral data obtained from performing reflectance spectroscopy may include intensity measurements of light reflection for various light wavenumbers. In another example, spectral data obtained from performing transmission spectroscopy may include intensity measurements of light transmission for various light wavenumbers. As an illustrative example, an intensity measurement may be a ratio of light intensity applied to light intensity absorbed, reflected, or transmitted for light at a wavenumber.

Although examples described herein may be discussed with reference to diagnosis of the SARS-CoV-2 virus, some embodiments may be used for diagnosis of other pathogens in a subject. Some embodiments may be used for diagnosis of any DNA or RNA virus. For example, some embodiments may be used for diagnosis of the Marburg virus, Ebola virus, rabies, human immunodeficiency virus (HIV), smallpox, hantavirus, influenza, dengue, rotavirus, severe acute respiratory syndrome (SARS), Middle East respiratory syndrome (MERS), human bocavirus 1, human coronavirus 229E, human coronavirus NL63, human coronavirus OC43, human enterovirus 68, human parainfluenza virus 1, human parainfluenza virus 4, rhinovirus 89, influenza A, influenza B, influenza H3N2 measles, mumps, SARS-CoV-1, or other pathogen. Some embodiments may be used for diagnosis of any viral pathogen, bacterial pathogen, fungal pathogen, parasitic pathogen, protozoan pathogen, or any pathogen that can be identified.

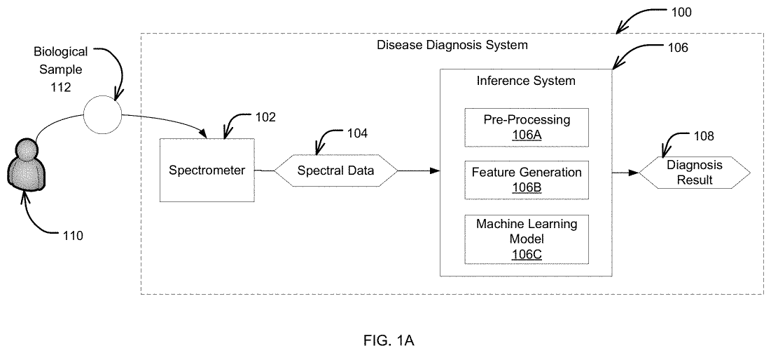

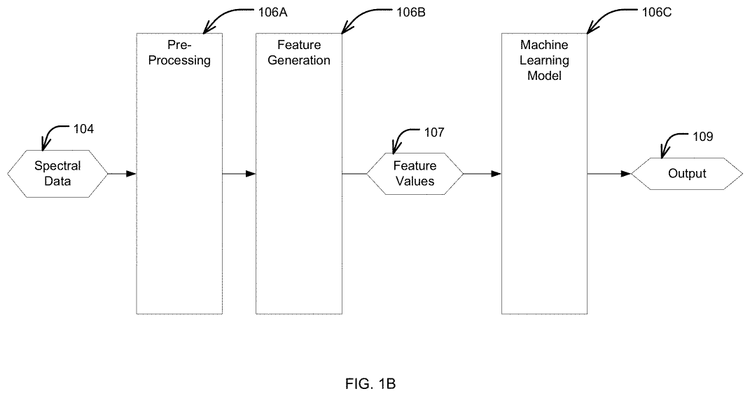

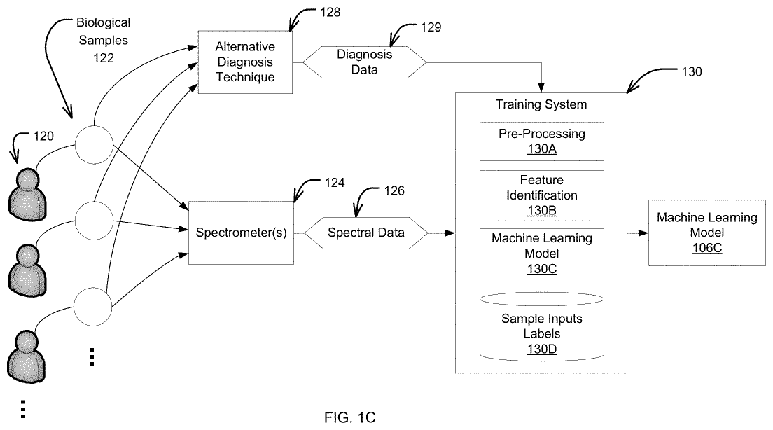

FIG. 1A illustrates an example disease diagnosis system 100, according to some embodiments of the technology described herein. As shown in FIG. 1A, the disease diagnosis system 100 receives a biological sample 112 from a subject 110 and determines a diagnosis result 108 for the subject 110. In some embodiments, the disease diagnosis system 100 may be configured to diagnose the COVID-19 virus in a subject. In some embodiments, the disease diagnosis system 100 may be configured to diagnose another pathogen in a subject. Examples pathogens are described herein.

As shown in FIG. 1A, a biological sample 112 is taken from a subject 110. In some embodiments, the biological sample 112 may be a portion of a blood sample, saliva sample, a nasal sample, a nasopharyngeal sample, urine sample, fecal sample, skin sample, hair sample, or any other suitable sample. As an illustrative example, the biological sample 112 may be a nasopharyngeal swab sample obtained from the subject 110 using a synthetic tip. The biological sample 112 from the subject 110 may be stored in a sterile container (e.g., a tube) containing transport media. For example, the sterile container may include VTM-N viral transport media developed by CITOSWAB.

In some embodiments, the biological sample 112 may be genetic material extracted from a sample taken from the subject. In some embodiments, the extracted genetic material may be an RNA extraction of a sample from the subject 110. As an illustrative example, the biological sample 112 may be an RNA extraction of a blood, saliva, nasal, or nasopharyngeal sample from the subject 110. The RNA extraction of the sample may be obtained using an RNA extraction kit. For example, the RNA extraction may be obtained using a GENRUI extraction kit. In some embodiments, the genetic material may be a DNA extraction of a sample from the subject 110. For example, the biological sample 112 may be a DNA extraction from a blood, saliva, or nasopharyngeal sample from the subject 110. The DNA extraction of the sample may be obtained using a DNA extraction kit. In some embodiments, the extracted genetic material may be proteins, antibodies, hormones or any other suitable genetic material.

As shown in FIG. 1A, the disease diagnosis system 100 includes a spectrometer 102 and an inference system 106.

In some embodiments, the spectrometer 102 may be configured to perform infrared (IR) spectroscopy on the biological sample 112. In some embodiments, the spectrometer 102 may be an emission spectrometer, an absorption spectrometer, a reflectance spectrometer, or a transmission spectrometer. In some embodiments, the spectrometer 102 may be an FTIR spectrometer. For example, the spectrometer 102 may be an attenuated total reflection (ATR) FTIR spectrometer (e.g., JASCO4600 ATR FTIR spectrometer). In some embodiments, the spectrometer 102 may be configured to perform X-ray spectroscopy, ultraviolet spectroscopy, or other suitable type of spectroscopy. In some embodiments, the spectrometer 102 may be configured to perform laser spectroscopy in which the spectrometer 102 uses a laser light as a radiation source.

In some embodiments, the spectrometer 102 may be configured to perform IR spectroscopy on the biological sample 112 by exposing the biological sample 112 to various wavelengths of light in an IR region of the light spectrum. For example, the spectrometer 102 may apply light beams of different wavelengths in the IR region to the biological sample 112. The spectrometer 102 may include a detector configured to measure an interaction of the light with molecules in the biological sample 112 (e.g., by measuring absorbance, reflectance, or transmission of different wavelengths of light by the biological sample 112). The spectrometer 102 may be configured to output spectral data 104 that comprises light intensity measurements for different light wavelengths (e.g., indicted by respective wavenumbers). For example, spectral data 104 may include, for each light wavenumber applied to the biological sample 112, a light intensity measurement of absorption, reflectance, or transmission of light of the wavenumber. As an illustrative example, a light intensity measurement may be a ratio or percentage indicative of absorption, reflectance, or transmission of light of the wavenumber.

In some embodiments, the spectrometer 102 may include a source. The source may be configured to generate radiation (e.g., light) that is directed to the biological sample 112. In some embodiments, the source may be configured to generate infrared (IR) radiation. For example, the source may generate radiation having wavelengths between 100 cm⁻¹ and 6000 cm⁻¹. In some embodiments, the source may be configured to generate a beam of IR light that is passed through an ATR crystal that is contact with the biological sample 112. The beam of IR light may reflect off the internal surface of the ATR crystal in contact with the biological sample 112. The reflection may form an evanescent wave that extends into the biological sample 112. The beam may be detected or measured (e.g., by a detector) when it exits the ATR crystal.

In some embodiments, the spectrometer 102 may include a detector. In some embodiments, the detector may be an infrared (IR) detector. The detector may be configured to measure an intensity of light incident at the detector. In some embodiments, the detector may be a pyroelectric detector. For example, the pyroelectric detector may be a deuterated lanthanum a-alanine doped triglycine sulphate (DLaTGS) pyroelectric detector. In some embodiments, the detector may be a thermal detector, photoconducting detector, or other suitable type of detector. Light (e.g., IR light) incident to the detector may cause electrical excitation in the detector. The detector may be configured to generate an electrical signal in response to light incident at the detector.

In some embodiments, the spectrometer 102 may be configured to process electrical signals generated by a detector to generate the spectral data 104. The spectrometer 102 may include an analog to digital converter configured to convert one or more electrical signals output by a detector into one or more digital signal(s). The spectrometer 102 may be configured to process the digital signal(s) to generate the spectral data 104. For example, the spectrometer 102 may determine a Fourier transform of the digital signal to generate the spectral data 104. In some embodiments, the spectrometer 102 may include a computing device in the spectrometer 102 for performing processing. For example, the computing device may include a processor and memory storing instructions that, when executed by the processor, cause the processor to determine a Fourier transform of a digital signal to generate the spectral data 104. Each of the light intensity measurements may indicate a ratio of light detected to light applied to the biological sample 112.

In some embodiments, the inference system 106 may be a computing device. For example, the inference system 106 may be a computing device communicatively coupled to the spectrometer 102. In some embodiments, the inference system 106 may be embedded within the spectrometer 102. For example, the inference system 106 may be implemented on a microcontroller in the spectrometer 102. In some embodiments, the inference system 106 may be separate from the spectrometer 102. For example, the inference system 106 may be a computing device in communication with the spectrometer 102. The inference system 106 may be a mobile device (e.g., smartphone, tablet, or a laptop computer), desktop computer, a server, or other suitable computing device. In some embodiments, the inference system 106 may be communicatively coupled to the spectrometer 102 by a physical connection (e.g., a wire). In some embodiments, the inference system 106 may be communicatively coupled to the spectrometer 102 by a wireless connection. In some embodiments, the inference system 106 may be remote from the spectrometer 102. For example, the inference system 106 may be communicatively coupled to the spectrometer 102 through a communication network (e.g., the Internet, or a local area connection (LAN)).

As shown in FIG. 1A, the inference system 106 is configured to receive spectral data 104 output by the spectrometer 102. The inference system 106 may be configured to use the spectral data 104 to generate the diagnosis result 108. The inference system 106 includes various components including a pre-processing module 106A, a feature generation module 106B, and a machine learning model 106C.

In some embodiments, the pre-processing module 106A may be configured to pre-process the spectral data 104 received by the inference system 106. In some embodiments, the pre-processing module 106A may be configured to apply filtering to the spectral data 104. For example, the pre-processing module 106A may apply a noise filter to the spectral data 104 to reduce the level of noise in the data. In some embodiments, the pre-processing module 106A may be configured to determine one or more derivatives of the spectral data 104. For example, the pre-processing module 106A may determine a first, second, and/or third derivative of the spectral data 104. In some embodiments, the pre-processing module 106A may be configured to apply smoothing to the spectral data 104 and/or a derivative thereof. For example, the pre-processing module may apply exponential smoothing, moving average smoothing, or other suitable type of smoothing. In some embodiments, the pre-processing module 106A may be configured to apply smoothing by applying a filter to the data (e.g., the spectral data 104, or a derivative thereof). For example, the pre-processing module 106A may apply a digital filter to the data. Example filters that may be used include a Savitzkey-Golay filter, a low pass filter, a mean filter, median filter, or other suitable filter.

In some embodiments, the pre-processing module 106A may be configured to apply a baseline correction to the spectral data 104. The pre-processing module 106A may be configured to apply the baseline correction by subtracting light intensity measurements of a baseline solvent. For example, the biological sample 112 may be placed in a baseline solvent of water. The pre-processing module 104 may be configured to subtract light intensity measurements determined for water from the spectral data 104. In some embodiments, the pre-processing module 106A may be configured to normalize the spectral data 104. For example, the pre-processing module 106A may normalize the light intensity measurements to a value between −1 and 1.

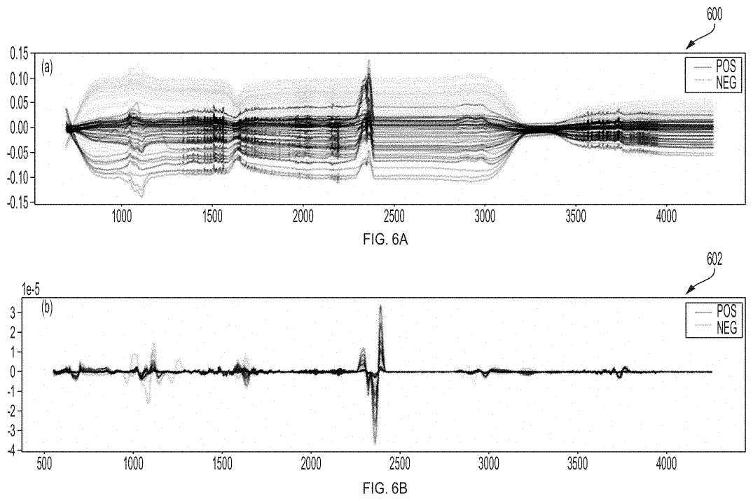

FIG. 6A is a graph 600 plotting spectral data obtained from performing IR spectroscopy on a biological sample. The graph 600 shows a light intensity measurement for light wavelengths ranging from 600 cm⁻¹ to 4500 cm⁻¹. In the example of FIG. 6A, the light intensity measurement for each of the wavelengths (e.g., wavenumbers) is a ratio of light intensity applied to the biological sample 112 to light intensity of reflected, absorbed, or transmitted light. As shown in FIG. 6A, the biological sample 112 has different levels of reflection for different wavenumbers. FIG. 6B is a graph 602 of the data of graph 600 after undergoing pre-processing, according to some embodiments of the technology described herein. Graph 602 is a second derivative taken of the spectral data plotted in graph 600 after applying filter (e.g., a Savitzky-Gola filter) to the spectral data plotted in graph 600.

In some embodiments, the feature generation module 106B may be configured to generate a set of feature values (e.g., to provide as input to the machine learning model 106C). The feature generation module 106B may be configured to use the spectral data 104 (e.g., after pre-processing by pre-processing module 106A) to generate the set of feature values. In some embodiments, the feature generation module 106B may be configured to determine the set of feature values to be a set of latent variables. For example, the latent variables may be principal components determined from performing principal component analysis (PCA) on a set of training data. In this example, the feature generation module may project the pre-processed spectral data into a principal component space (e.g., using eigenvectors determined from performing PCA) to obtain the set of feature values. In another example, the latent variables may be predictors determined from performing partial least squares regression (PLS) on a set of training data. In this example, the feature generation module 106B may project the spectral data 104 into a latent variable space determined from performing PLS. In another example, the latent variables may be a set of variables output by a layer of a neural network (e.g., an encoder of an auto-encoder). In this example, the feature generation module 106B may provide the spectral data 104 as input to the neural network to obtain values output by the layer.

In some embodiments, the feature generation module 106B may be configured to generate the set of feature values using the pre-processed spectral data by: (1) selecting a subset of wavelengths of the spectral data 104; and (2) generating the set of feature values from the subset of wavelengths to generate the set of feature values. The subset of light wavelengths may be determined to provide a spectral signature of a pathogen (e.g., COVID-19) which is being diagnosed by the system 100. For example, when the pathogen is present in the biological sample 112, values (e.g., light intensity values or a derivative thereof) for the subset of light wavelengths (e.g., in spectral data and/or pre-processed spectral data) may meet one or more patterns (e.g., that may be recognized by machine learning model 106C). In another example, spectral data for the subset of light wavelengths may meet one or more signal shapes. In some embodiments, the subset of light wavelengths may be determined by applying optimization techniques to a set of training data to identify a subset of light wavelengths that may be used for diagnosis of a disease. For example, the subset of light wavelengths may be determined by performing mixed integer optimization to learn a subset of light wavelengths that indicate a spectral signature of a pathogen (e.g., COVID-19).

In some embodiments, the feature generation module 106B may be configured to determine the values for the subset of light wavelengths in the pre-processed spectral data to be the set of feature values. For example, the feature generation module 106B may determine values of a first or second derivative of the spectral data at the subset of light wavelengths to be the set of feature values. In another example, the feature generation module 106B may determine values of normalized and/or filtered spectral data at the subset of wavelengths to be the set of feature values. In some embodiments, the feature generation module 106B may be configured to use the values for the subset of light wavelengths to generate the set of feature values. For example, the feature generation module 106B may determine one or more linear combinations of the values for the subset of light wavelengths to be the set of feature values.

In some embodiments, the inference system 106 may be configured to provide a generated set of feature values as input to a machine learning model 106C. The machine learning model 106C may be trained to output an indication of whether a pathogen (e.g., SARS-CoV-2) is present in the biological sample 112. In some embodiments, the machine learning model 106C may be trained to output a classification indicating whether the pathogen is present in the biological sample 112. For example, the machine learning model 106C may be configured to output a binary classification indicating that: (1) the pathogen is present in the biological sample 112; or (2) the pathogen is not present in the biological sample 112. In some embodiments, the machine learning model 106C may be trained to output a value indicative of a likelihood (e.g., probability) that the pathogen is present in the biological sample 112. For example, the machine learning model 106C may output a value between 0 and 1 indicative of the likelihood that the pathogen is present in the biological sample 112.

In some embodiments, the inference system 106 may be configured to determine the diagnosis result 108 based on the output of the machine learning model 106C. For example, the inference system 106 may determine that the subject 110 is diagnosed with a virus when the machine learning model 106C outputs a classification indicating that the pathogen is present in the biological sample 112. The inference system 106 may determine that the subject 110 is not diagnosed with the virus when the machine learning model 106C outputs a classification indicating that the pathogen is not present in the biological sample 112. In another example, the inference system 106 may determine the diagnosis result 108 based on an indication of likelihood that the pathogen is present in the biological sample 112 output by the machine learning model 106C. For example, the system may determine that the subject 110 is diagnosed with a virus when the indication of the likelihood exceeds a first threshold likelihood (e.g., 0.5, 0.6, 0.7, 0.8, 0.9, or 0.95), and that the subject 110 is not diagnosed with the virus when the indication of likelihood is below a second threshold likelihood (e.g., 0.3, 0.4, 0.5, 0.6, 0.7, or 0.8). In some embodiments, the first and second threshold likelihood may be the same. In some embodiments, the inference system 106 may be configured to determine an inconclusive diagnosis result 108. For example, the machine learning model 106C may output a classification indicating that there was no conclusion about the presence of a pathogen in the biological sample 112. In another example, the machine learning model 106C may output an indication of a likelihood that is between a first threshold for a positive diagnosis and a second threshold for a negative diagnosis.

As an illustrative example, the inference system 106 may determine the diagnosis result 108 to be that: (1) the subject 110 is COVID-19 positive when the machine learning model 106C outputs a prediction (e.g., a classification) indicating that SARS-CoV-2 is present in the biological sample 112; and (2) the subject 110 is COVID-19 negative when the machine learning model 106C outputs a prediction (e.g., classification) indicating that SARS-CoV-2 is not present in the biological sample 112. In some embodiments, the inference system 106 may be configured to determine the diagnosis result 108 based on an output indicating a likelihood (e.g., a probability) that SARS-CoV-2 is present in the biological sample 112. The inference system 106 may be configured to determine the diagnosis result 108 by determining the subject 110 to be COVID-19 positive when the value exceeds a threshold likelihood, and to not be COVID-19 negative when the value is less than the threshold likelihood.

In some embodiments, the machine learning model 106C may comprise a set of parameters (e.g., learned during training) that are stored by the inference system 106. The inference system 106 may be configured to use the machine learning model 106C by providing a set of feature values as input to the machine learning model 106C. The inference system 106 may determine an output of the machine learning model by performing computations using the set of feature values and learned parameters. The inference system 106 may be configured to store the parameters in memory of the inference system 106. The inference system 106 may be configured to use the stored parameters to determine an output of the machine learning model 106C for an input set of feature values. For example, the inference system 106 may perform computations using learned parameters of the machine learning model 106C to determine an output value (e.g., a classification).

In some embodiments, the machine learning model 106C may be a support vector machine (SVM). In some embodiments, the machine learning model 106C may be a logistic regression model. In some embodiments, the machine learning model 106C may be a neural network (NN). For example, the machine learning model 106C may be a convolutional neural network (CNN), a recurrent neural network (RNN), or other suitable type of neural network. In some embodiments, the machine learning model 106C may be a decision tree model. In some embodiments, the machine learning model 106C may be a Naïve Bayes classifier.

FIG. 1B illustrates a data flow diagram through components of the inference system 106 of FIG. 1A, according to some embodiments of the technology described herein. As shown in FIG. 1B, the spectral data 104 (e.g., received from the spectrometer 102) is processed by the pre-processing module 106A. The pre-processed spectral data 104 is then provided to the feature generation module 106B. The feature generation module 106B generates a set of feature values 107 that are provided as input to the machine learning model 106C. The machine learning model 106C generates an output 109 (e.g., a classification, or likelihood value) based on which the inference system 106 generates the diagnosis result 108. In some embodiments, the output 109 of the machine learning model 106C may be the diagnosis result 108.

FIG. 1C illustrates an example of a training system 130 for training a machine learning model 130C to obtain a trained machine learning model 106C used by the disease diagnosis system 100 of FIG. 1A, according to some embodiments of the technology described herein. As shown in FIG. 1C, the training system 130 receives spectral data 126 obtained by one or more spectrometers 124, and diagnosis data 129 determined from an alternative diagnosis technique 128. The training system 130 uses the spectral data 126 and the diagnosis data 129 to output trained machine learning model 106C described herein with reference to FIG. 1A.

As shown in FIG. 1C, the spectrometer(s) 124 may be used to perform spectroscopy (e.g., IR spectroscopy) on biological samples 122 (e.g., nasal, saliva samples, or genetic material extractions therefrom) taken from multiple different subjects 120. Example spectrometers and biological samples are described herein with reference to FIG. 1A. For example, each of the spectrometer(s) 124 may be spectrometer 102 described herein with reference to FIG. 1A, and each of the biological samples 122 may be as described with reference to biological sample 112 of FIG. 1A.

As shown in FIG. 1C, the biological samples 122 may also be analyzed by an alternative diagnosis technique 128 to determine a diagnosis. The diagnosis data 129 may include diagnosis results as determined by the alternative diagnosis technique 128. For example, the alternative diagnosis technique 128 used for a COVID-19 diagnosis system may be an RT-PCR based test. The diagnosis data 129 from performing alternative diagnosis technique 128 may be indications of whether each of the subjects 120 is determined to have a pathogen (e.g., SARS-CoV-2) based on the alternative diagnosis technique 128. For example, the diagnosis data 129 may include an identifier for each of the biological samples 122, and a binary value indicating whether the sample is determined to include the pathogen.

As shown in FIG. 1C, the training system 130 includes multiple components including a pre-processing module 130A, a feature identification module 130B, an untrained machine learning model 130C, and a datastore 130D storing sample inputs and corresponding labels.

In some embodiments, the pre-processing module 130A may be configured to pre-process the spectral data 126 as described with respect to pre-processing module 106A of inference system 106, described herein with reference to FIG. 1A. The pre-processing module 106A may be configured to: (1) obtain the spectral data 126 obtained from performing spectroscopy on each of the biological samples 122; and (2) pre-process the spectral data for each biological sample 122 to generate sample inputs. Each of the sample inputs may represent a respective one of the one of the biological samples 122 obtained from a respective one of the subject2 120. The pre-processing module 130A may be configured to store the sample inputs in the datastore 130D. The sample inputs may be used as part of a training data set for training the machine learning model 130C.

In some embodiments, the pre-processing module 130A may be configured to label the training data set. The pre-processing module 130A may be configured to label the training data set by, for each sample input: (1) determining a diagnosis indicated by the diagnosis data 129; and (2) assign a label to the set of data according to the diagnosis. For example, the system may assign a binary value (e.g., 0 or 1) indicating whether the sample input corresponds to a biological sample determined to have a pathogen present in it. The labels assigned to the data sets may represent target outputs to use in training a machine learning model (e.g., using supervised learning techniques). As shown in FIG. 1C, the pre-processing module 130A may be configured to store the labels in the datastore 130D.

In some embodiments, the feature identification module 130B may be configured to determine a set of features to use as input to the machine learning model 130C. The feature identification module 130B may be configured to determine the set of features by analyzing a training data set (e.g., of sample inputs and labels stored in datastore 130D). In some embodiments, the determine set of features may have a lower dimensionality than that of the spectral data 126. For example, spectral data for a sample input may include light intensity measurements for thousands of wavelengths. Having a number of features that is greater than the number of samples in the data set may degrade performance (e.g., accuracy) of a machine learning model. Using all the light intensity measurements across all the light wavelengths may thus limit performance of the machine learning model in predicting whether a subject is infected with a disease. Moreover, using all the wavelengths would increase the number of parameters in a machine learning model, and thus the computational resources needed to use the machine learning model (e.g., during inference). Accordingly, the feature identification module 130B may be configured to determine a set of variables that has a reduced dimensionality relative to the spectral data.

In some embodiments, the feature identification module 130B may be configured to determine the set of features by determining a set of latent variables to use as the set of feature values that are provided as input to the machine learning model 130C. In some embodiments, the feature identification module 130B may be configured to apply principal component analysis (PCA) on the training data set to determine the set of latent variables. For example, the feature identification module 130B may apply PCA on the training data set to determine one or more vectors to use for transforming a spectral data sample into a set of latent variables in a principal component space. In some embodiments, the feature identification module 130B may be configured to apply partial least squares (PLS) regression on the training data to determine the set of latent variables. For example, the feature identification module 130B may apply PLS on the training data set to determine one or more vectors to use for transforming a spectral data sample into a set of latent variables in a principal component space. In some embodiments, the system may be configured to generate a set of latent variables using a neural network. For example, the system may train an auto-encoder, and use an encoder of the auto-encoder to generate the set of latent variables representing a sample of spectral data.

In some embodiments, the feature identification module 130B may be configured to generate the set of features by identifying a set of light wavelengths that indicate a spectral signature for a pathogen. The set of light wavelengths may be a subset of light wavelengths of spectral data obtained from performing spectroscopy on a biological sample. For example, the feature identification module 130B may identify a subset of light wavelengths of the spectral data that provide a spectral signature of COVID-19. Values of spectral data or pre-processed spectral data for the subset of light wavelengths may then be used as the set of feature values, or to generate the set of feature values for input to the machine learning model 130C.

In some embodiments, the feature identification module 130B may be configured to identify a subset of light wavelengths that indicate a spectral signature for a pathogen by performing mixed integer optimization. By performing mixed integer optimization, the feature identification module 130B may identify spectral values (e.g., intensity and/or shape) for a specified number of light wavelengths as a set of features. In some embodiments, the feature identification module 130B may be configured to perform sparse mixed integer optimization to identify the set of light wavelengths. For example, the feature identification module 130B may use techniques described in “Novel Mixed Integer Optimization Sparse Regression Approach in Chemometrics,” published in Analytica Chimica Acta volume 1137, pages 115-124, in September 2020, which is incorporated by reference herein in its entirety. The determined subset of wavelengths may be indicative of characteristics or processes in the biological sample. For example, different wavelengths may represent different chemical characteristics and/or processes in a biological sample. The values for the subset of wavelengths may be interpretable (e.g., by a clinician) to determine a cause of a diagnosis result.

In some embodiments, techniques described in the reference may be used to build a classification model that uses light intensity measurements for a subset of light wavelengths. In some embodiments, the subset of light wavelengths may consist of less than 5, 10, 15, 20, 25, 30, 35, 40, 45, 50, 55, 60, 65, 70, 75, 80, 85, 90, 95, 100, 200, 300, 400, or 500 light wavelengths. In some embodiments, the subset of light wavelengths may consist of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, or 500 light wavelengths. In some embodiments, the subset of light wavelengths may consist of any number between 1-200 of light wavelengths.

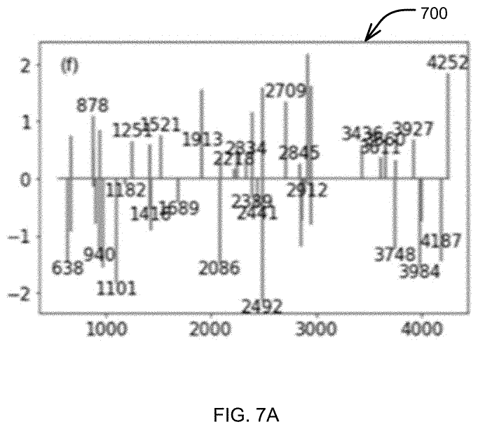

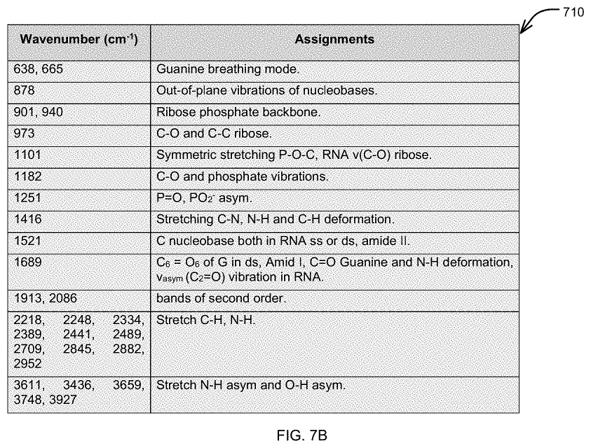

FIG. 7A is a graph 700 of a subset of light wavelengths of spectral data used to generate a set of feature values for input to a machine learning model, according to some embodiments of the technology described herein. The subset of light wavelengths shown in the graph 700 of FIG. 7A are selected using sparse mixed integer optimization. As shown in FIG. 7A, a subset of approximately 47 wavelengths have been selected from a range of wavenumbers from 600 cm⁻¹ to 4500 cm⁻¹. The graph 700 displays, for each of the subset of light wavelengths, a value of a second derivative of the spectral data plotted in graph 600 of FIG. 6A.

FIG. 7B is a table 710 listing characteristic and/or processes associated with the light wavelengths of FIG. 7A, according to some embodiments of the technology described herein. As shown in table 710, each wavelength (indicated by a wavenumber) from FIG. 7A has an associated chemical characteristic. For example, wavenumbers 638 cm⁻¹ and 665 cm⁻¹ may represent Guanin breathing mode, wavenumber 878 cm⁻¹ may represent out-of-plane vibrations of nucleobases, and wavenumber 1182 cm⁻¹ may represent carbon monoxide and phosphate vibrations. Light intensity measurements for these wavelengths may thus provide an indication of characteristics and/or processes of a biological sample which may facilitate interpreting a diagnosis result generated using a machine learning model.

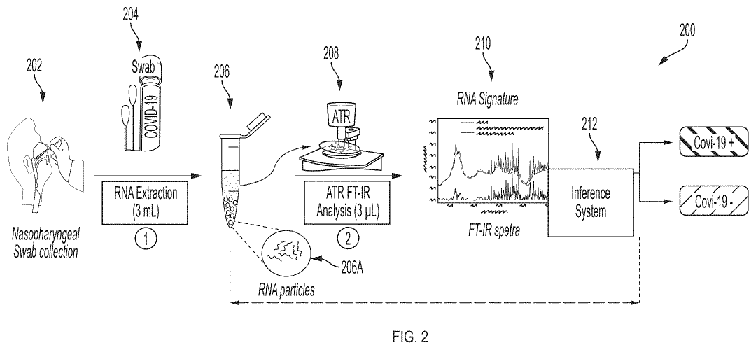

FIG. 2 is a diagram of an example process 200 for diagnosing COVID-19 in a subject, according to some embodiments of the technology described herein. The process 200 may be implemented using disease diagnosis system 100 described herein with reference to FIGS. 1A-B.

As shown in the example of FIG. 2, a nasopharyngeal swab sample is obtained from a subject 202. At step 204, a gene material (e.g., RNA) extraction is performed on the swab sample to obtain an RNA extraction sample 206. The RNA extraction sample 206 may include RNA particles 206A of the subject in the extraction sample 206. At step 208, a spectrometer is used to perform spectroscopy on a portion of the RNA extraction sample 206. In the example of FIG. 2, the spectrometer performs ATR FTIR spectroscopy on the portion of the RNA extraction sample. The spectrometer generates spectral data 210 from the spectroscopy. The spectral data 210 may comprise light intensity measurements for multiple different light wavelengths. An inference system 212 (e.g., which may be inference system 106 described herein with reference to FIG. 1A-B) may be used to generate a diagnosis result using the spectral data 210. The inference system 212 outputs a diagnosis result of the subject 202 being positive for COVID-19 (e.g., that SARS-CoV-2 is present in the subject), or negative for COVID-19 (e.g., that SARS-CoV-2 is not present in the subject).

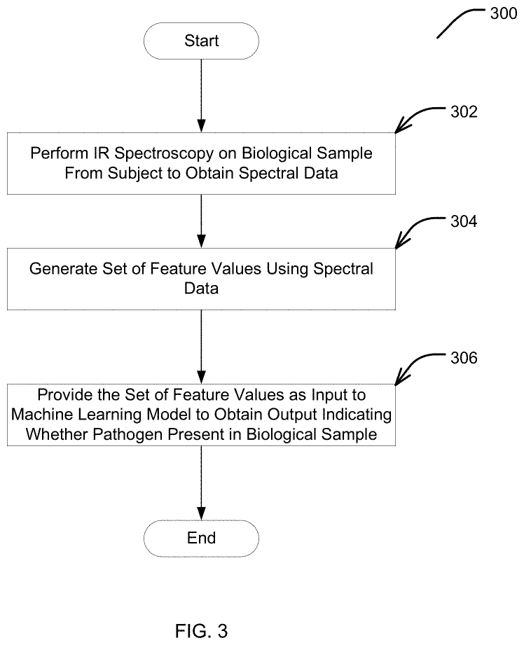

FIG. 3 is a flowchart of an example process 300 for diagnosing whether a pathogen is present a subject, according to some embodiments of the technology described herein. In some embodiments, process 300 may be performed by disease diagnosis system 100 described herein with reference to FIGS. 1A-B. In some embodiments, process 300 may be performed to diagnose COVID-19 in a subject. In some embodiments, process 300 may be performed to perform a diagnosis of another pathogen. Examples of disease are described herein.

Process 300 begins at block 302, where the system performing process 300 performs IR spectroscopy on a biological sample from a subject to obtain spectral data. The biological sample may be biological sample 112 described herein with reference to FIG. 1A. For example, the biological sample may be a nasal, saliva, blood, or other suitable sample from the subject. In another example, the biological sample may be a sample of genetic material (e.g., RNA or DNA) extracted from a sample (e.g., nasal, saliva, or blood sample) obtained from the subject.

In some embodiments, the system may be configured to perform IR spectroscopy on the biological sample using a spectrometer to obtain spectral data. For example, the system may use spectrometer 102 described herein with reference FIG. 1A. In some embodiments, the system may perform IR spectroscopy to generate the spectral data. The spectral data may be spectral data 104 described herein with reference to FIGS. 1A-B. For example, the spectral data may be obtained by applying a Fourier transform to one or more digital signals indicative of light intensity measured by a detector of the spectrometer.

The spectral data may include light intensity measurements for multiple different light wavelengths (e.g., in an IR spectrum). In some embodiments, the spectral data may include light intensity measurements for light wavelengths in a range of approximately 10 cm⁻¹ to 14,000 cm⁻¹, 100 cm⁻¹ to 14000 cm⁻¹, 200 cm⁻¹ to 13000 cm⁻¹, 300 cm⁻¹ to 12000 cm⁻¹, 400 cm⁻¹ to 11000 cm⁻¹, 500 cm⁻¹ to 10000 cm⁻¹, 600 cm⁻¹ to 9000 cm⁻¹, 600 cm⁻¹ to 8000 cm⁻¹, 600 cm⁻¹ to 7000 cm⁻¹, 600 cm⁻¹ to 6000 cm⁻¹, 600 cm⁻¹ to 6000 cm⁻¹, 600 cm⁻¹ to 5000 cm⁻¹, 600 cm⁻¹ to 4500 cm⁻¹, 800 cm⁻¹ to 2000 cm⁻¹, 900 cm⁻¹ to 1800 cm⁻¹, or any suitable range within any one of these ranges. In some embodiments the spectral data may include light intensity measurements for the wavelengths at a resolution of 0.1 cm⁻¹, 1 cm⁻¹, 2 cm⁻¹, 3 cm⁻¹, 4 cm⁻¹, 5 cm⁻¹, 10 cm⁻¹, or other suitable resolution. In some embodiments, a light intensity measurement for a wavelength may be a measure of reflectance, absorbance, or transmittance of light of the wavelength (e.g., determined by the spectrometer).

Next, process 300 proceeds to block 304, where the system generates a set of feature values using the spectral data. The system may be configured to generate the set of feature values using the spectral data by pre-processing the spectral data (e.g., second derivative values determined after applying filtering to the spectral data). In some embodiments, the system may be configured to use light intensity measurements to generate the set of feature values by: (1) determine a set of latent variables using the light intensity measurements; and (2) determining the set of latent variables to be the set of feature values. The latent variables may be used to generate a set of feature values with lower number of dimensions than the spectral data. For example, the spectral data may have light intensity measurements for thousands of wavelengths. The system may use the latent variables to generate a set of feature values. In some embodiments, the system may be configured to determine the set of latent variables to be principal components determined from performing PCA or PLS on a training data set. For example, the system may determine the principal components by using a set of one or more eigenvectors obtained from performing PCA or PLS to obtain a feature vector. In another example, the system may determine a linear combination of one or more light intensity measurements determined from performing linear discriminant analysis (LDA) on a set of training data to generate the set of feature values.

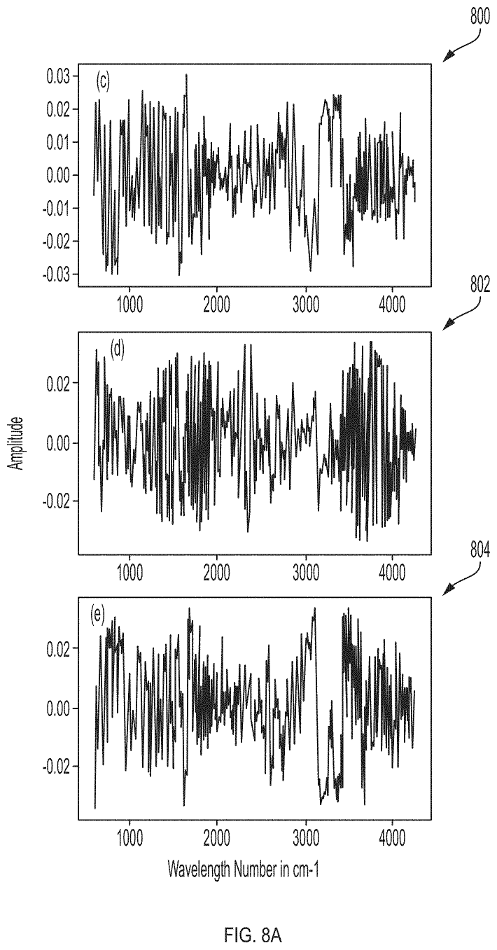

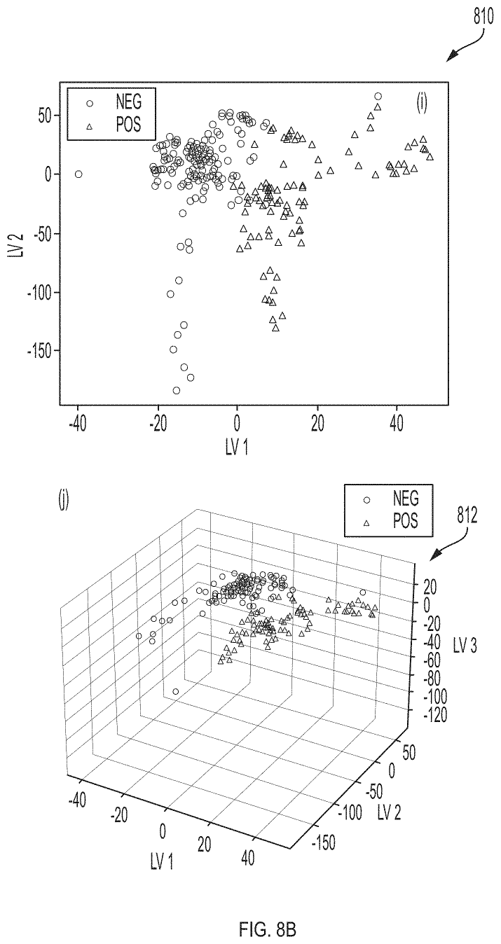

FIG. 8A is a set of graphs 800, 802, 804 of latent variables to use as feature values input to a machine learning model, according to some embodiments of the technology described herein. Each of the graphs 800, 802, 804 shows a respective latent variable determined from performing partial least squares regression discriminant analysis (PLS-DA) on a set of training data. Each of the graphs 800, 802, 804 shows a plot of a latent variable with respect to wavelength. FIG. 8B is a set of graphs of projections of the latent variables of FIG. 8A, according to some embodiments of the technology described herein. Graph 810 is a projection of different sets of spectral data obtained from different subjects according to the latent variables plotted in graphs 800, 802 of FIG. 8A. Graph 812 is a projection of different sets of spectral data obtained from different subjects according to the latent variables plotted in graphs 800, 802, 804 of FIG. 8A.

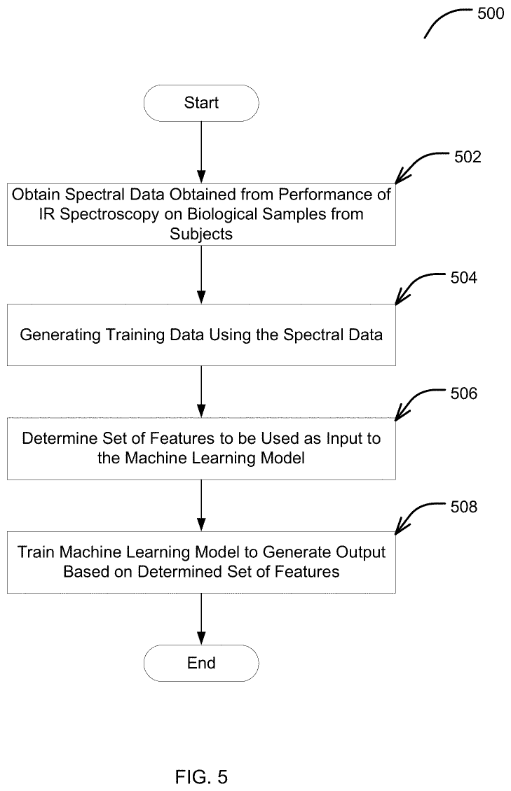

Next, process 300 proceeds to block 306, where the system provides the set of feature values as input to a machine learning model (e.g., a logistic regression model, an SVM model, neural network model, or other type of model) to obtain output indicating whether a pathogen is present in the biological sample. The machine learning model may be trained to output an indication of whether the pathogen is in the biological sample. Example techniques for training the machine learning model are described herein with references to FIGS. 1C and 5. As an illustrative example, the machine learning model may be trained to output a classification (e.g., a binary classification) of whether the pathogen is present in the biological sample. As another example, the machine learning model may be trained to output a value indicating a likelihood (e.g., a probability) that the pathogen is present in the biological sample.

In some embodiments, the system may be configured to use the output of the machine learning model to determine a diagnosis result. For example, if the machine learning model outputs a classification that the pathogen (e.g., SARS-CoV-2) is in the biological sample, the system may output a positive diagnosis result (e.g., COVID-19 positive). If the machine learning model outputs a classification that the pathogen is not in the biological sample, the system may output a negative diagnosis result (e.g., COVID-19 negative). In another example, the machine learning model may output an indication of a likelihood that the subject is infected with the disease. The system may be configured to determine a diagnosis result based on the indication of the likelihood. The system may be configured to output a positive diagnosis result when the indication is above a threshold likelihood and a negative diagnosis result when the indication is below a threshold likelihood. In some embodiments, the system may be configured to output a diagnosis result indicating that the diagnosis is inconclusive (e.g., if the indication of the likelihood falls in between a positive threshold likelihood and a negative threshold likelihood).

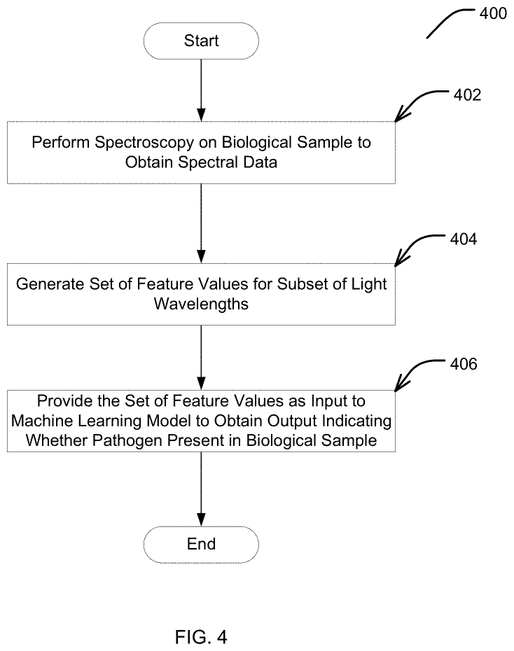

FIG. 4 is a flowchart of an example process 400 for diagnosing whether a pathogen is present in a subject, according to some embodiments of the technology described herein. In some embodiments, process 400 may be performed by disease diagnosis system 100 described herein with reference to FIGS. 1A-B. In some embodiments, process 400 may be performed to diagnose COVID-19 in the subject. In some embodiments, process 400 may be performed to perform a diagnosis whether another pathogen is present in a subject. Examples of pathogens are described herein.

Process 400 begins at block 402, where the system performing process 400 performs spectroscopy on a biological sample from a subject to generate spectral data. The system may perform spectroscopy on the biological sample to generate the spectral data as described at block 302 of process 300 described herein with reference to FIG. 3.

In some embodiments, the system may be configured to perform spectroscopy on the biological sample using a spectrometer to obtain spectral data. For example, the system may use spectrometer 102 described herein with reference FIG. 1A. In some embodiments, the system may perform IR spectroscopy to generate the spectral data. The spectral data may be spectral data 104 described herein with reference to FIGS. 1A-B. For example, the spectral data may be obtained by applying a Fourier transform to one or more digital signals indicative of light intensity measured by a detector of the spectrometer.

The spectral data may include light intensity measurements for multiple different wavelengths of lights (e.g., in an IR spectrum). In some embodiments, the spectral data may include light intensity measurements for light wavelengths in a range of approximately 350 cm⁻¹ to 7800 cm⁻¹, 600 cm⁻¹ to 8000 cm⁻¹, 10 cm⁻¹ to 14,000 cm⁻¹, or any suitable range within any one of these ranges. In some embodiments the spectral data may include light intensity measurements for the light wavelengths at a resolution of 0.1 cm⁻¹, 1 cm⁻¹, 2 cm⁻¹, 3 cm⁻¹, 4 cm⁻¹, 5 cm⁻¹, 10 cm⁻¹, or other suitable resolution.

In some embodiments, a light intensity measurement for a light wavelength may be a measure of reflectance, absorbance, or transmittance of light of the light wavelength (e.g., measured by a spectrometer). In some embodiments, the light intensity measurement may be a ratio of light applied to light measured at a detector. For example, the light intensity measurement may be a ratio indicating a reflectance of light of the wavelength by the biological sample.

Next, process 400 proceeds to block 404, where the system generates a set of feature values for a subset of wavelengths (e.g., wavenumbers) of the spectral data. In some embodiments, the system may be configured to generate the set of feature values by determining the light intensity measurements for the subset of light wavelengths to be set of feature values. In some embodiments, the system may be configured to generate the set of feature values for the subset of wavelengths by: (1) pre-processing the spectral data; and (2) determining pre-processed values determined for the subset of light wavelengths to be the set of feature values. In some embodiments, the system may be configured to pre-process the data by determining a derivative (e.g., a first derivative, second derivative, or a third derivative) of the spectral data. The system may determine the values of the derivative at the subset of wavelengths to be set of feature values. For example, the system may determine a second derivative of the spectral data and determine values of the second derivative at the subset of wavelengths to be the set of feature values. In some embodiments, the system may be configured to pre-process the data by applying filtering and/or smoothing to the spectral data. Example techniques by which the system may perform pre-processing as described in reference to pre-processing module 106A described herein with reference to FIGS. 1A-B.

In some embodiments, the subset of light wavelengths for which the system determines values may be a subset of light wavelengths that are determined to provide a spectral signature of a disease. For example, the subset of light wavelengths may be determined to provide a spectral signature of COVID-19. When the pathogen is present in a biological sample, the set of feature values for the subset of light wavelengths may meet one or more patterns. In some embodiments, the subset of wavelengths may be determined in a training stage for training a machine learning model. In some embodiments, the subset of wavelengths may be determined by applying mixed integer optimization to a set of training data to identify the subset of light wavelengths. Example techniques for identifying the subset of light wavelengths are described herein with reference to the feature identification module 140B of FIG. 1C.

In some embodiments, the system may be configured to generate the set of feature values for the subset of light wavelengths by applying a transformation to values of the spectral data or pre-processed spectral data at the subset of wavelengths. For example, the system may: (1) provide the values determined for the subset of light wavelengths as input to a function to obtain one or more corresponding output values; and (2) use the output value(s) as the set of feature values.

Next, process 400 proceeds to block 406, where the system provides the set of feature values as input to a machine learning model (e.g., a logistic regression model, an SVM model, neural network model, or other type of model) to obtain output indicating whether a pathogen is present in the biological sample. The machine learning model may be trained to output an indication of whether the pathogen is present in the biological sample. Example techniques for training the machine learning model are described herein with references to FIGS. 1C and 5. As an illustrative example, the machine learning model may be trained to output an indication (e.g., a binary value) of a classification of whether the pathogen is present in the biological sample. As another example, the machine learning model may be trained to output a value indicating a likelihood (e.g., a probability) that the pathogen is present in the biological sample.

In some embodiments, the system may be configured to use the output of the machine learning model to determine a diagnosis result. For example, if the machine learning model outputs a classification that the pathogen (e.g., SARS-CoV-2) is in the biological sample, the system may output a positive diagnosis result (e.g., COVID-19 positive). If the machine learning model outputs a classification that the pathogen is not in the biological sample, the system may output a negative diagnosis result (e.g., COVID-19 negative). In another example, the machine learning model may output an indication of a likelihood that the subject is infected with the disease. The system may be configured to determine a diagnosis result based on the indication of the likelihood. The system may be configured to output a positive diagnosis result when the indication is above a threshold likelihood and a negative diagnosis result when the indication is below a threshold likelihood. In some embodiments, the system may be configured to output a diagnosis result indicating that the diagnosis is inconclusive (e.g., if the indication of the likelihood falls in between a positive threshold likelihood and a negative threshold likelihood).

In some embodiments, the machine learning model may be trained to recognize a spectral signature of a pathogen. The spectral signature of the pathogen may be one or more patterns of the set of feature values indicating that the pathogen is present in the biological sample. The machine learning model may be trained to recognize the pattern(s). An example process for training the machine learning model is described herein with reference to FIG. 5.

FIG. 5 is a flowchart of an example process 500 for training a machine learning model for diagnosing whether a pathogen is present in a subject, according to some embodiments of the technology described herein. For example, the machine learning model may be a logistic regression model, support vector machine (SVM), neural network, or other suitable machine learning model. In some embodiments, process 500 may be performed to train a machine learning model for diagnosing whether SARS-CoV-2 is present in a subject. In some embodiments, process 500 may be performed to train a machine learning model for diagnosing whether another pathogen is present in the subject. Example pathogens are described herein. Process 500 may be performed by training system 130 described herein with reference to FIG. 1C. For example, process 500 may be performed to obtained machine learning model 106C used by disease diagnosis system 100 described herein with reference to FIGS. 1A-B.

Process 500 begins at block 502, where the system obtains data obtained from performance of IR spectroscopy on biological samples from subjects. The IR spectroscopy may be performed as described at block 302 of process 300 described herein with reference to FIG. 3. The spectral data may include, for each of the subjects, light intensity measurements (e.g., of absorbance, transmission, or reflectance) for wavelengths of light (e.g., wavenumbers).

Next, process 500 proceeds to block 504, where the system generates training data using the spectral data. In some embodiments, the system may be configured to generate the training data by pre-processing the spectral data. For example, the system may pre-process the spectral data as described herein with reference to pre-processing module 130A of training system 130 described herein with reference to FIG. 1C. For example, the system may pre-process the spectral data by: (1) applying filtering (e.g., Savitzky-Golay filtering) to the spectral data; and (2) determining a first or second derivative of the spectral data. In another example, the system may pre-process the spectral data by normalizing the spectral data. In another example, the system may pre-process the spectral data by applying baseline correction to the spectral data (e.g., by subtracting baseline light intensity measurements from those of the spectral data). In some embodiments, the system may be configured to pre-process the spectral data by performing any combination of one or more pre-processing techniques described herein.

In some embodiments, the system may be configured to generate the training data by determining labels for the training data. The system may be configured to label each of the spectral data samples obtained from performing IR spectroscopy on respective biological samples. The system may be configured to label each spectral data sample as indicating that a pathogen (e.g., SARS-CoV-2) is present in a respective biological sample (e.g., with a binary value of 1) or that the pathogen is not present (e.g., with a binary value of 0). In some embodiments, the system may be configured to determine the labels based on diagnosis data obtained from an alternative diagnosis technique. For example, the system may use diagnosis data obtained from performing an RT-PCR based test for presence of SARS-CoV-2 in the biological samples. In this example, the system may label each of the spectral data samples as positive (e.g., with a value of 1) or negative (e.g., with a value of 0) for SARS-CoV-2 based on the diagnosis from the RT-PCR based test.

Next, process 500 proceeds to block 506, where the system determines a set of features to be used as input to the machine learning model. In some embodiments, the system may be configured to determine a set of features that have a number of dimensions that is less than the number of wavelengths in a spectral data samle. For example, a spectral data sample may include light intensity measurements for over 8,000 wavelengths of light (e.g., wavenumbers). However, the number of samples may be less than the number of wavelengths of light. For example, the number of samples may be less than 100, 200, 300, 400, or 500 samples. Determining features for all the wavelengths in the spectral data may hinder performance of the machine learning model. Moreover, a machine learning model that uses an input set of features with thousands of dimensions requires more computational resources (e.g., time and energy) and may be less efficient to use during inference. Accordingly, the system may determine a set of features with a fewer number of dimensions than that of the spectral data. Example numbers of dimensions are described herein.

In some embodiments, the system may be configured to determine the set of features by determining a subset of wavelengths of the spectral data that indicate a spectral signature of the pathogen. The machine learning model may thus be trained to recognize whether the spectral signature is present in a biological sample of a subject based on the subset of wavelengths. Example sizes of the subset of wavelengths are described herein. The system may be configured to determine the set of features to be values for the subset of wavelengths (e.g., in spectral data or pre-processed spectral data). For example, the set of features of may be values of a derivative (e.g., a first or second derivative) of light intensity measurements of spectral data at the subset of wavelengths. In some embodiments, the set of features may be light intensity measurements of the spectral data (e.g., before or after pre-processing). In some embodiments, the set of features may be values derived from values for the subset of wavelengths. For example, the set of features may include one or more linear combinations of the values.

In some embodiments, the system may be configured to determine the subset of wavelengths by performing mixed integer optimization to identify the subset of wavelengths. For example, the system may use techniques described in “Novel Mixed Integer Optimization Sparse Regression Approach in Chemometrics,” published in Analytica Chimica Acta volume 1137, pages 115-124, in September 2020. In this example, given a data matrix X that represents the spectral data, and a response vector Y representing an output of the machine learning model, a loss function L, and a regularization function it, the techniques may be used to build the machine learning model by solving equation 1 below.

Min_{B}L(Y,X,β)+yπ(β), s. t.|β∥₀≤k Equation 1

In equation 1, y is a non-negative parameter, k is a positive integer, and ∥·∥₀ is the L₀ norm indicating the number of non-zero variables in β. In some embodiments, the loss function may be a sigmoid function. In some embodiments, the regularization function may be Tikhonov regularization function.

In some embodiments, the system may be configured to determine the set of features by determining a set of latent variables as the set of features. In some embodiments, the system may be configured to determine the set of latent variables by performing principal component analysis (PCA) on the training data. The system may be configured to perform PCA to identify one or more principal components along which the system may orient spectral data (e.g., after pre-processing). In some embodiments, the system may be configured to determine the set of latent variables by performing partial least squares (PLS) regression on the training data to determine the set of latent variables. In some embodiments, the system may be configured to train a neural network and use an output of a layer of the neural network as the set of latent variables. For example, the system may train an auto-encoder, and use an output of the encoder of the trained auto-encoder as the set of latent variables. In some embodiments, the system may be configured to perform multi-dimensional scaling (MDS), isometric feature mapping (Isomap), locally linear embedding (LLE), Hessian eigenmapping (HLLE), spectral embedding (Laplacian Eigenmaps), t-distributed stochastic neighbor embedding (t-SNE), or other suitable dimension reduction technique to determine the set of features.

After determining the set of features at block 506, process 500 proceeds to block 508, where the system trains the machine learning model to generate an output based on the determined set of features. The system may be configured to: (1) for each of the spectral data samples, determine values of the set of features; and (2) train the machine learning model using the sets of feature values. In some embodiments, the system may be configured to train the machine learning model by applying a supervised learning technique to the sets of feature values and corresponding labels. For example, the system may perform stochastic gradient descent to train the machine learning model. In this example, the system may iteratively provide the sets of feature values as input to the machine learning model to obtain an output (e.g., a classification). The system may: (1) determine a measure of difference between the target labels, and the outputs; and (2) update parameters of the machine learning model based on the difference. The system may determine a gradient of a loss function based on the output of the machine learning model, and update the parameters based on the gradient. For example, the system may use a mean squared error (MSE) loss, binary cross-entropy loss, or other suitable loss function.

In some embodiments, the system may be configured to train the machine learning model using an unsupervised learning technique (e.g., when the sets of feature values are unlabeled). The system may be configured to apply a clustering algorithm to the sets of feature values to cluster the samples into positive and negative results. For example, the system may apply k-means clustering to determine clusters. As an illustrative example, for implementations in which the machine learning model is to diagnose presence of SARS-CoV-2 in a subject, the system may determine a cluster indicating that SARS-CoV-2 is not present in a biological sample, and a second cluster indicating that SARS-CoV-2 is present in the biological sample.

In some embodiments, where the set of feature values are values for a subset of wavelengths of light in a spectral data sample, the machine learning model may be trained to recognize a spectral signature of a pathogen indicated by the subset of wavelengths. The subset of wavelengths may adhere to one or more patterns when a pathogen (e.g., SARS-CoV-2) is present in a biological sample. The system may be configured to train the machine learning model to recognize the pattern(s). For example, the system may train the machine learning model to recognize the pattern(s) by applying supervised or unsupervised learning techniques to a set of training data.

In some embodiments, the system may be configured to train the machine learning model by further tuning one or more hyperparameters of the machine learning model. For example, the system may tune a solver, regularization, and/or penalty (the “C parameter”) for a logistic regression model. In another example, the system may tune a kernel and/or penalty of an SVM. In another example, the system may tune a learning rate, number of hidden layers, and/or activation function for a neural network. In some embodiments, the system may be configured to tune one or more hyperparameters of the machine learning model by performing cross-validation. The system may be configured to use a percentage (e.g., approximately 67%) of the sets of feature values for training, and the remaining sets of feature values for testing. As an illustrative example, for a set of 280 sets of feature values, the system may use 185 sets of feature values for training, and 95 sets of feature values for testing. The system may be configured to assess statistical significance by shuffling the training and testing sets of feature values a number of times. For example, the system may shuffle the training and testing sets of feature values 25 times.

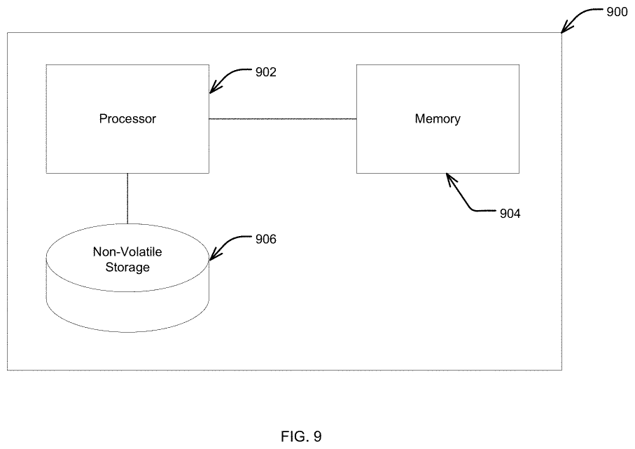

FIG. 9 is an illustrative implementation of a computer system 900 that may be used in connection with some embodiments of the technology described herein. The computing device 900 may include one or more computer hardware processors 902 and non-transitory computer-readable storage media (e.g., memory 904 and one or more non-volatile storage devices 906). The processor(s) 902 may control writing data to and reading data from (1) the memory 904; and (2) the non-volatile storage device(s) 906. To perform any of the functionality described herein, the processor(s) 902 may execute one or more processor-executable instructions stored in one or more non-transitory computer-readable storage media (e.g., the memory 904), which may serve as non-transitory computer-readable storage media storing processor-executable instructions for execution by the processor(s) 902.

The terms “program” or “software” or “module” are used herein in a generic sense to refer to any type of computer code or set of processor-executable instructions that can be employed to program a computer or other processor (physical or virtual) to implement various aspects of embodiments as discussed above. Additionally, according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform tasks or implement abstract data types. Typically, the functionality of the program modules may be combined or distributed.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments.

### Example Implementation

Some embodiments of techniques described herein were tested on a sample of 280 symptomatic and asymptomatic subjects. Among the subjects, 100 were determined to be COVID-19 positive and 180 were determined to be COVID-19 negative based on a RT-PCR test. COVID-19 positive. Swab samples were obtained from the subjects, and RNA extractions were obtained from the swab samples. The RNA extraction samples were analyzed by ATR IR spectroscopy. The obtained spectral data was then used to train and test a machine learning model. The machine learning model indicated results with 97.8% accuracy, 97% sensitivity, and 98.3% specificity. The spectral data indicates the presence of three wavelength domains located at 600-1350 cm⁻¹, at 1500-1700 cm⁻¹ and at 2300-3900 cm⁻¹ attributable to an RNA fingerprint of COVID-19 (e.g., i.e., phosphate backbone vibrations (νP-O), νC-O stretching vibrations of ribose sugar, and the specific RNA nucleobases). The region 2400-3900 cm⁻¹ may be attributed to the stretching vibrations of OH, NH, and CH groups.

Nasopharyngeal swab samples were collected from the subjects using swabs with a synthetic tip. Swabs were immediately inserted into sterile tubes containing 1-3 mL of viral transport media. Extraction kits from different vendors (e.g., APMLIX, MOLARRAY, BIOER and GENRUI) were used for RNA extraction. 100 mL of viral transport media was added to the kit, while the remaining purification process was fully automated by the extractor in Viral Mode. The sample output was of 50 μL.

To perform a real-time RT-PCR diagnosis, TAKYON REAL-TIME ONE-STEP RT-PCR MASTER MIX and EUROGENETIC kit was used. Each 25 μL reaction mixture contained 12.5 μL of 2×reaction buffer, 1 mL of forward and reverse primers at 10 mM, 0.5 mL of probe at 10 mL, 0.25 RTenzyme, 0.5 RNase inhibitor, and 5 μL of RNA template. Amplification was carried out in 96-well plates on QUANTSTUDIO 1 machine developed by THERMOFISHER SCIENTIFIC. Thermocycling conditions consist of 55° C. for 10 minutes for reverse transcription, followed by 95° C. for 3 minutes and then 45 cycles of 95° C. for 15 seconds and 58° C. for 30 seconds. Each run included one SARS-CoV-2 genomic template control and one no-template control for the PCR-amplification step. For a routine workflow, the E gene assay was carried out as the first-line screening tool followed by confirmatory testing with the EUROGENETIC RdRp gene assay. Positive samples for both E gene assay and RdRp assay should had a cycle threshold CT value lower than 35. Results for E gene with CT value greater than 35 was confirmed with the RdRp assay.

For performing ATR FTIR spectroscopy, a JASCO 4600 ATR-FTIR spectrometer with a deuterated lanthanum a-alanine doped triglycine sulphate (DLaTGS) pyroelectric detector. The detector was operated with temperature stabilization using electrical Peltier temperature control. The spectrometer was paired with a high-intensity ceramic light source. Reflection ATR was performed using high-throughput monolithic diamond crystal and 64 spectra were averaged. A torque limiter pressure was applied for reproducible sample pressure contact for sample measurements. Distilled water was used as a solvent background. 3 μL of each sample were spread on the ATR crystal, ensuring that no air bubbles were trapped. Samples were not dried on as it may increase the testing time, at the expense of having to deal with absorption from water. After the acquisitions, the crystal was cleaned with ethanol (70% v/v) and dried using paper towel. Spectral data was collected for wavenumbers ranging between 600 cm⁻¹−8000 cm⁻¹ with a spectral resolution of 0.7 cm⁻¹. In some embodiments, the wavenumbers ranging from 900 cm⁻¹ to 1800 cm⁻¹ region may be an RNA bio fingerprint region.

A Logistic regression, SVM, Kernel SVM and Discriminant machine learning model were trained for the implementation. A quarter of the training data was used for cross-validation to tune the hyperparameters of the machine learning models.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having,” “containing,” “involving,” and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A method of training a machine learning model for diagnosing whether a pathogen is present in a subject, the method comprising:
using a processor to perform:
obtaining spectral data obtained from performing IR spectroscopy on biological samples obtained from a plurality of subjects, wherein the spectral data comprises, for each of the plurality of subjects, light intensity measurements for a plurality of wavelengths of light;
generating a set of training data using the spectral data; and
training the machine learning model using the training data, the training comprising determining a set of features for the machine learning model, wherein the set of features has a number of dimensions that is less than a number of the plurality wavelengths.

2. The method of claim 1, wherein determining the set of features comprises determining a subset of wavelengths of the plurality of wavelengths that indicate a spectral signature of the pathogen.

3. The method of claim 2, wherein determining the subset of the plurality of wavelengths to be the set of features comprises determining less than 100 of the plurality of wavelengths to be the set of features.

4. The method of claim 2, further comprising determining the subset of wavelengths at least in part by performing mixed integer optimization to identify the subset of wavelengths.

5. The method of claim 1, wherein determining the set of features comprises performing principal component analysis (PCA) to identify the set of features.

6. The method of claim 1, wherein determining the set of features comprises performing partial least square (PLS) regression to identify the set the features.

7. The method of claim 1 comprising:
obtaining diagnosis data comprising, for each of the plurality of subjects, an indication of whether the pathogen is determined to be present in the subject based on a different diagnosis technique; and
generating the set of training data by using the diagnosis data to label sets of feature values for the at least some subjects.

8. The method of claim 1, wherein the pathogen is SARS-CoV-2.

9. The method of claim 1, wherein the machine learning model comprises a logistic regression model.

10. The method of claim 1, wherein the plurality of wavelengths of light range from approximately 600 cm⁻¹ to 4500 cm⁻¹.

11. The method of claim 1, wherein the biological samples comprise extractions of genetic materials.

12. The method of claim 1, wherein determining the set of features for the machine learning model comprises:
determining a second derivative of the spectral data; and
determining the set of features using the second derivative values.

13. The method of claim 12, wherein processing the spectral data comprises applying Savitzky-Golay filtering to the spectral data.

14. A system of training a machine learning model for diagnosing whether a pathogen is present in a subject, the system comprising:
a processor; and
a non-transitory computer-readable storage medium storing instructions, that when executed by the processor, causes the processor to perform:
obtaining spectral data obtained from performing IR spectroscopy on biological samples obtained from a plurality of subjects, wherein the spectral data comprises, for each of the plurality of subjects, light intensity measurements for a plurality of wavelengths of light; and
training the machine learning model using the spectral data, the training comprising determining a set of features for the machine learning model, wherein the set of features has a number of dimensions that is less than a number of the plurality wavelengths.

15. The system of claim 14, wherein determining the set of features comprises determining a subset of wavelengths of the plurality of wavelengths that indicate a spectral signature of the pathogen.

16. The system of claim 15, wherein the instructions further cause the processor to perform identifying the subset of wavelengths at least in part by performing mixed integer optimization to identify the subset of wavelengths.

17. The system of claim 14, wherein the pathogen is SARS-CoV-2.

18. The system of claim 14, wherein the plurality of wavelengths range from approximately 600 cm⁻¹ to 4500 cm⁻¹.

19. The system of claim 14, wherein the biological samples comprise extractions of genetic materials.

20. A non-transitory computer-readable storage medium storing instructions that, when executed by a processor, cause the processor to perform a method to train a machine learning model for diagnosing whether a pathogen is present in a subject, the method comprising:
obtaining spectral data obtained from performing IR spectroscopy on biological samples obtained from a plurality of subjects, wherein the spectral data comprises, for each of the plurality of subjects, light intensity measurements for a plurality of wavelengths of light; and
training the machine learning model using the spectral data, the training comprising determining a set of features for the machine learning model, wherein the set of features has a number of dimensions that is less than a number of the plurality wavelengths.
